(19) United States
(12) Patent Application Publication
(10) Pub. No.: US 2026/0294318 A1
(43) Pub. Date: Oct. 1, 2026

(54) System and Method for Performing a Therapeutic and/or Diagnostic Function in a Patient

(71) Applicant: BIOTRONIK SE & Co., KG, Berlin (DE)

(72) Inventors: R. Hollis WHITTINGTON, Portland, OR (US); Patrick L. PARISH, Portland, OR (US); Dirk MUESSIG, West Linn, OR (US); Brian M. TAFF, Portland, OR (US)

(73) Assignee: BIOTRONIK SE & Co., KG, Berlin (DE)

(21) Appl. No.: 19/480,489

(22) PCT Filed: Apr. 23, 2024
(86) PCT No.: PCT/EP2024/061032

§ 371 Date: Oct. 31, 2025

Related U.S. Application Data
(60) Provisional application No. 63/466,380, filed on May 15, 2023.

(30) Foreign Application Priority Data
Jun. 7, 2023 (EP) 23177964.6

(51) Int. Cl. A61B 5/358; A61B 5/00; A61B 5/287

(52) U.S. Cl. CPC A61B 5/358; A61B 5/0006; A61B 5/287; A61B 5/746

(57) ABSTRACT

A system for performing a therapeutic and/or diagnostic function in a patient includes an implantable medical device configured for implantation in a patient, the implantable medical device including an electrode arrangement having a multiplicity of electrode poles for sensing an electrocardiogram signal. A processing arrangement is configured to process the electrocardiogram signal to derive information from the electrocardiogram signal relating to an ST segment subsequent to an R peak of a QRS complex and prior to a T wave in a cardiac cycle and to identify a myocardial infarction event based on the information.

### CROSS-REFERENCE TO RELATED APPLICATIONS

This application is the United States National Phase under 35 U.S.C. § 371 of PCT International Patent Application No. PCT/EP2024/061032, filed on Apr. 23, 2024, which claims the benefit of European Patent Application No. 23177964.6, filed on Jun. 7, 2023, and U.S. Provisional Patent Application No. 63/466,380, filed on May 15, 2023, the disclosures of which are hereby incorporated by reference herein in their entireties.

### TECHNICAL FIELD

The instant invention generally relates to a system for performing a therapeutic and/or diagnostic function in a patient and to a method for operating such a system.

### BACKGROUND

A system of the type concerned herein comprises an implantable medical device configured for implantation in a patient, the implantable medical device comprising an electrode arrangement having a multiplicity of electrode poles for sensing an electrocardiogram signal. A processing arrangement is configured for processing the electrocardiogram signal obtained by the electrode arrangement.

An implantable medical device of this kind may for example be a pacemaker, an implantable cardioverter defibrillator, a sensor device such as a bio-sensor, or a monitoring device. The implantable medical device herein is configured to sense electrocardiogram signals.

For example, the implantable medical device may be a monitoring device which is configured to record electrocardiogram signals and to communicate recorded electrocardiogram signals or information derived from recorded electrocardiogram signals to an external device in the context of a home monitoring system.

An implantable medical device as e.g. used in a home monitoring system shall allow for a reliable monitoring of a physiological state of a patient. In particular, using the implantable medical device it shall be possible to reliably detect an abnormal cardiac state. If an abnormality is detected, the implantable medical device shall be enabled to communicate with for example an external device of a home monitoring system, e.g. in order to trigger a message to a service center to alert medical personnel of a potential need for attention.

Among those cardiac abnormalities which make a close, reliable monitoring desirable is a state of an acute myocardial infarction. Acute myocardial infarctions represent a leading cause of death in the developed world resulting in, nowadays, an estimated 3,000,000 lives lost worldwide per year with more than 1,000,000 lives lost in the US alone. As in a state of acute myocardial infarction immediate medical attention to a patient is crucial to improve the chances of treating the myocardial infarction, it is desired to improve approaches to support predicting, recognizing and rapidly reporting the occurrence of sudden ischemic cardiac conditions.

Outside of clinical settings, few tools and/or devices exist to assist patients in their acute needs for interfacing with the health care system and/or receiving catered, time-critical care in response to myocardial infarctions. Many such circumstances that aren't immediately fatal often, unfortunately, become so simply because the means to respond has often leaned on strategies demanding that the patient, bystanders, or their caregiver take action to notify potentially responsive medical personnel.

The present disclosure is directed toward overcoming one or more of the above-mentioned problems, though not necessarily limited to embodiments that do.

### SUMMARY

It is an object of the instant invention to provide a system for performing a therapeutic and/or diagnostic function in a patient and a method for operating such a system which allow for a monitoring of a cardiac condition to identify a myocardial infarction event in a timely manner.

In one aspect, a system for performing a therapeutic and/or diagnostic function in a patient comprises an implantable medical device configured for implantation in a patient, the implantable medical device comprising an electrode arrangement having a multiplicity of electrode poles for sensing an electrocardiogram signal. The system furthermore comprises a processing arrangement for processing the electrocardiogram signal obtained by the electrode arrangement. The processing arrangement is configured to process the electrocardiogram signal to derive information from the electrocardiogram signal relating to an ST segment subsequent to an R peak of a QRS complex and prior to a T wave in a cardiac cycle and to identify a myocardial infarction event based on the information.

The system comprises an implantable medical device configured for implantation in a patient. The implantable medical device comprises an electrode arrangement configured for sensing electrocardiogram signals. To implement the electrode arrangement, the implantable medical device may for example comprise one or multiple leads, for example extending from a generator, each lead carrying one or multiple electrode poles. The generator herein may be for example configured for implantation external to the patient's heart, for example at a subcutaneous location. The one or multiple leads may extend from the generator and may reach into the patient's heart, such that electrode poles may be arranged within the heart, for example in the right or left ventricle or within the right or left atrium. In other embodiments, the electrode arrangement may be implemented by electrode poles arranged on a device housing.

The implantable medical device may for example be a pacemaker, an implantable cardioverter defibrillator, a sensor device such as a bio-sensor, or a monitoring device.

The processing arrangement is configured to process the electrocardiogram signal obtained by the electrode arrangement to derive information from the electrocardiogram signal relating to an ST segment subsequent to an R peak of a QRS complex and prior to a T wave in a cardiac cycle. The QRS complex and the T wave of a cardiac cycle may be identified within the electrocardiogram signal, the QRS complex for example exhibiting peaks denoted as Q peak, R peak and S peak. The ST segment is defined as a signal portion extending in between a QRS complex and a subsequent T wave of a cardiac cycle and may be identified for example by signal analysis, for example by identifying the end of a QRS complex, or based on a timing distance with respect to a QRS complex and/or a T wave.

It generally has been observed that, in case of a myocardial infarction event, a signal shape in the ST segment between a QRS complex and a subsequent T wave in a cardiac cycle is altered in comparison to a signal shape in a healthy cardiac state. In a healthy cardiac state, without the presence of a myocardial infarction, it generally can be assumed that the signal in the ST segment in between a QRS complex at a subsequent T wave in a cardiac cycle is close to an isoelectric baseline. The ST segment represents the interval between ventricular depolarization (represented by the QRS complex) and repolarization (represented by the T wave). In a healthy cardiac state, the signal in the ST segment is flat and close to the isoelectric baseline. In contrast, on the occasion of a myocardial infarction event, due to myocardial ischemia the signal shape in the ST segment will change, causing an ST segment elevation or depression and hence a significant deviation of the signal in the ST segment from the baseline.

It herein is proposed to sense electrocardiogram signals using an electrode arrangement of an implantable medical device and to process such electrocardiogram signals in order to derive information from the electrocardiogram signals relating to an ST segment and to identify a myocardial infarction event based on the information. By the processing, in particular, a deviation of a signal correlated with the ST segment from the isoelectric baseline may be identified, and based on this a myocardial infarction event may be identified.

In one embodiment, the implantable medical device comprises a processing circuitry which implements at least a portion of the processing arrangement.

In general, the processing may be conducted entirely within the implantable medical device in order to identify a myocardial infarction event by the implantable medical device. In this case the processing arrangement is implemented by the processing circuitry of the implantable medical device.

In other embodiments, the processing arrangement is in part or (almost) entirely implemented externally to the implantable medical device, in which case the implantable medical device for example only pre-processes an electrocardiogram signal obtained by the electrode arrangement and forwards the electrocardiogram signal to the processing arrangement external to the implantable medical device. The processing for identifying a myocardial infarction event hence takes place substantially outside of the implantable medical device, for example in an external device communicating with the implantable medical device for example in the context of a home monitoring system, or on a server device stationed at a remote location, such as a home monitoring service center connected to the external device and via the external device to the implantable medical device via a public communication network, such as the Internet.

In one embodiment, the system comprises an external device configured for operation external to the patient. The implantable medical device is configured to communicate data relating to the electrocardiogram signal or relating to information derived from the electrocardiogram signal to the external device. For example, the implantable medical device may communicate a portion of the electrocardiogram signal as such as a raw data signal to the external device for processing in the external device or in another device connected to the external device. In other embodiments, the implantable medical device may substantially carry out the processing for identifying a myocardial infarction event and may forward information relating to an identified myocardial infarction event to the external device, in which case at least a majority of the processing is carried out by the implantable medical device and only a post-processing is carried out by the external device or by a device connected to the external device.

In one embodiment, the system comprises a server device configured to communicate with the external device for transferring information to and/or receiving information from the external device. The server device, for example a home monitoring service center in the context of a home monitoring system, is configured to process information derived from the electrocardiogram signal to identify a myocardial infarction event and/or to process information relating to an identified myocardial infarction event. A substantial amount of the processing may be carried out by the server device, in which case for example raw data of the electrocardiogram signal or pre-processed information indicative of information derived from the electrocardiogram signal is forwarded to the server device, which carries out further processing for identifying a myocardial infarction event. In another embodiment, the server device may receive information relating to an identified myocardial infarction event, as it has been determined by the implantable medical device or by the external device, in order to post-process such information, for example to trigger an action in response to the identification of a myocardial infarction event.

In one embodiment, the multiplicity of electrode poles of the electrode arrangement form at least two pairs of electrode poles, the at least two pairs of electrode poles defining different signal reception vectors. For example, the electrode arrangement may be formed by one or multiple leads carrying a multiplicity of electrode poles, the electrode poles forming two or more pairs of electrode poles. One pair of electrode poles herein may be formed by electrode poles of a particular lead. Another pair of electrode poles may be formed by an electrode pole on one lead and another electrode pole on a different lead. Yet another pair of electrode poles may be formed by an electrode pole on a lead and another electrode pole formed by the housing of a generator device of the implantable medical device. The different pairs of electrode poles span different signal reception vectors, the different signal reception vectors spanning across different spatial regions of the heart and being oriented, preferably, along different spatial directions. By means of the different signal reception vectors spanned by the different pairs of electrode poles, hence, different electrocardiogram signals reflecting cardiac activity of different cardiac regions may be obtained.

By obtaining electrocardiogram signals using different signal reception vectors a multi-channel processing of electrocardiogram signals is enabled. In particular, electrocardiogram signals obtained by different signal reception vectors may be processed independently in order to derive information relating to an ST segment in a cardiac cycle as represented in the different signals, such that a combined processing of multi-channel electrocardiogram signals becomes possible in order to identify a myocardial infarction event.

In one embodiment, the processing arrangement is configured to process signals obtained by the at least two pairs of electrode poles to derive, for each signal reception vector, information relating to an ST segment. Hence, each electrocardiogram signal obtained by a particular signal reception vector spanned by a particular pair of electrode poles is processed in order to derive information relating to an ST segment as represented in the particular signal, wherein the information obtained according to the different signal reception vectors may be combined in order to identify a myocardial infarction event.

By processing signals according to the different signal reception vectors in parallel, it may become possible to derive spatially distinct information. In particular, it can be identified what cardiac region potentially is affected by ischemic conditions, based on an ST segment elevation or depression identified in one or some signals in comparison to other signals.

In one embodiment, the processing arrangement is configured, for deriving the information relating to an ST segment, to identify a signal portion relating to an ST segment based on a timing with respect to a QRS complex in a cardiac cycle. Generally, it is assumed that the ST segment in an electrocardiogram signal starts at the end of the S peak of the QRS complex, wherein regularly an angle may occur where the S peak transits into the comparatively flat signal portion of the ST segment. The junction point of the S peak and the ST segment also is denoted as J point. If however an ST elevation or depression occurs, it may not be easy to identify the J point based a signal analysis aimed at detecting a sharp angle representing the end of the S peak. In this case for example a timing may be used, for example with respect to the R peak of the QRS complex, wherein it for example can be assumed that the ST segment starts at a particular timing distance with respect to the prior R peak and lasts for a set duration.

The timing distance between an R peak and the start of the ST segment may for example be set to lie in a range between 0 ms and 150 ms, wherein the timing distance may depend on the heart rate and may be adapted based on the heart rate. The time duration of the ST segment may for example be set to lie in a range between 100 ms and 400 ms and may also be adaptive based on the heart rate.

A signal portion representing the ST segment may hence be extracted from an electrocardiogram signal by identifying the R peak of a QRS complex and by assuming a particular timing distance between the R peak and the start of the ST segment, and by further assuming a defined duration of the ST segment.

In one embodiment, the implantable medical device is configured to store at least one of a signal portion relating to an ST segment and information derived from the electrocardiogram signal relating to an ST segment. For example, if a substantial amount of the processing is carried out not by the implantable medical device, but by an external device or a server device connected to the external device, the implantable medical device may communicate a raw data signal or pre-processed information to the external device for further processing. If a substantial amount of the processing is carried out by the implantable medical device and the implantable medical device is enabled to identify a myocardial infarction event, the implantable medical device may store a signal portion relating to an identified myocardial infarction event, for example representing a QRS complex and a subsequent ST segment relating to the identified infarction event, and may communicate the signal portion together with identified information about the myocardial infarction event to an external device.

Generally, the monitoring for a myocardial infarction event may be carried out on every cardiac cycle or on a selection of some number of cardiac cycles or based on a periodic selection of cycles, such as in one cardiac cycle every 10 seconds, every 30 seconds, every minute, every 10 minutes, or the like. By performing e.g. a periodic monitoring in one cardiac cycle per defined time period, a computational load in particular in the implantable medical device may be reduced. The defined time period herein beneficially is chosen such that a sufficiently fast response in the occasion of a myocardial infarction event is enabled.

In one embodiment, the processing arrangement is configured to determine, in order to derive information from the electrocardiogram signal, a set of characteristic values from the electrocardiogram signal relating to an ST segment. Based on the characteristic values, e.g. based on a comparison of the characteristic values to a set of reference values defined for a healthy cardiac state, a myocardial infarction event may be identified. For example, based on an electrocardiogram signal, an amplitude of an ST segment elevation or depression, an integral under a curve relating to an ST segment, an amplitude at a number of points in an ST segment, a first-order and/or higher-order derivative value at one or multiple points in an ST segment and other values according to other defined metrics may be determined. Characteristic values as determined for the electrocardiogram signal may e.g. be compared to a set of corresponding reference values corresponding to a healthy cardiac state. If it is found that one, some or all of the characteristic values deviate from the set of reference values e.g. by more than a certain margin, a myocardial infarction event may be identified.

In one embodiment, the processing arrangement is configured to identify a myocardial infarction event based on the electrocardiogram signal using an artificial intelligence model. Using an artificial intelligence model, information may be derived from an electrocardiogram signal sensed by the electrode arrangement of the implantable medical device, and based on a processing by the artificial intelligence model it may be determined, based on the electrocardiogram signal, if a myocardial infarction event is present. For the processing, the electrocardiogram signal as such or information derived from the electrocardiogram signal, for example characteristic values relating to an ST segment, may be fed as input data into the artificial intelligence model. The artificial intelligence model processes input values and outputs an indication whether a myocardial infarction event is present. The indication herein may for example be a score value indicating a likelihood of the presence of a myocardial infarction event, wherein a large score value may indicate a high likelihood of a myocardial infarction event.

The artificial intelligence model may be implemented on a processing circuitry of the implantable medical device. In other embodiments the artificial intelligence model may be implemented on an external device or on a remote server device.

The artificial intelligence model may be a fixed model which is trained and defined in a training phase beforehand, prior to installation in the system. In other embodiments, the artificial intelligence model may be adaptive in that the model constantly adapts itself during operation based on an input of values obtained within the system and obtained from other sources.

The advantages of using an artificial intelligence model are that the right criteria to diagnose a myocardial infarction can be recognized, that the model can be adaptive, easily updated, and easily accessed on a server.

The artificial intelligence model might also identify the pertinent features instead of only extracting them manually and feeding to the artificial intelligence model. Like it can train on numerous waveforms and outcomes (patients who had MI) and determine what are the important actors that predict an MI in the ST segment data. This can overcome patient to patient and device variations.

For example, at a start the artificial intelligence model may be designed such that it exhibits a high sensitivity (indicating the probability of a positive test result if the patient truly suffers from a myocardial infarction) and reasonable specificity (indicating the probability of a negative test result if the patient truly does not suffer from a myocardial infarction), wherein the model may be adaptive during operation such that the specificity is improved.

In one embodiment, the processing arrangement is configured to train the artificial intelligence model using training data, the training data including information obtained from a multiplicity of implantable medical devices. The artificial intelligence model may, in one embodiment, be trained in an initial training phase based on a set of data from a database of infarct and non-infarct data. During operation of the system, data may be collected from a large amount of implantable medical devices implanted in a large variety of patients, wherein the data may be fed into the model and may be used to further train the model in order to refine the model for improving its sensitivity and/or for improving its specificity.

An amount of training data useful for training the model hence may evolve over time subject to analytics applied to ongoing data collection efforts applied across a pool of patients implanted with such implantable medical devices who experienced known and confirmed myocardial infarction events. The collection of information may be supported by any one of multiple possible avenues including but not limited to mechanisms that relay the status of patient and device conditions to wearables, mobile devices, or bedside units that can pass data further on to centralized repositories. Artificial intelligence-based surveys of a growing body of information collected from the pool of patients may serve to enhance the predictability and accuracy of the model for identifying a myocardial infarction event.

In one embodiment, the processing arrangement is configured to trigger an alert action based on the identification of a myocardial infarction event. Generally, if a myocardial infarction event is identified, the processing arrangement may trigger an alert message for example to healthcare personnel in order to alert the healthcare personnel of a worsening of the condition of the patient.

For example, for triggering an alert action, the processing arrangement may be configured to generate, as the alert action, an alarm message to an emergency facility, the message including information for identifying the patient to the emergency facility. For example, an automatic call to an emergency hotline, e.g. an emergency phone number such as “911” in the US, may be initiated, such that an emergency facility is alarmed to provide immediate attention to the patient suffering from an acute myocardial infraction. A message to the emergency facility may for example include the time of identification of the myocardial infarction event, the name of the patient and a physical location of the patient, such that emergency treatment may be provided to the patient.

In addition, a message for example to identified individuals, such as family members or friends may be triggered in order to alert such individuals.

In another aspect, a method for operating a system for performing a therapeutic and/or diagnostic function in a patient comprises: providing an implantable medical device configured for implantation in a patient, the implantable medical device comprising an electrode arrangement having a multiplicity of electrode poles for sensing an electrocardiogram signal; and processing the electrocardiogram signal obtained by the electrode arrangement, including processing the electrocardiogram signal to derive information from the electrocardiogram signal relating to an ST segment subsequent to an R peak of a QRS complex and prior to a T wave in a cardiac cycle and identifying a myocardial infarction event based on the information.

Another aspect aims to drive adaptive responses to the therapy delivery in implanted devices facilitating near-immediate, proactive therapy delivery responses in the interim period before a more robust set of diagnostics and remediations can be brought to bear in a clinical setting. It is the aim to develop a capability within implanted devices to adapt their stimulation output in ways that avoid implant-recognized ischemic regions within the heart tissue following myocardial infarction. This adaptive response, offers a means to proactively improve on the support offered to patients encountering such episodes in the interim between the event itself and the time when more comprehensive assessments and care can be administered in a clinical setting.

An embodiment may be a system for performing a therapeutic and/or diagnostic function in a patient, comprising:
an implantable medical device configured for implantation in a patient, the implantable medical device comprising an electrode arrangement having a multiplicity of electrode poles for sensing an electrocardiogram signal; and a processing arrangement for processing the electrocardiogram signal obtained by the electrode arrangement; wherein the processing arrangement is configured to process the electrocardiogram signal to derive information from the electrocardiogram signal relating to an ST segment subsequent to an R peak of a QRS complex and prior to a T wave in a cardiac cycle and to identify a myocardial infarction event based on the information; wherein the multiplicity of electrode poles form at least two pairs of electrode poles, the at least two pairs of electrode poles defining different signal reception vectors, and wherein the processing arrangement is configured to process signals obtained by the at least two pairs of electrode poles to derive, for each signal reception vector, information relating to an ST segment to identify a myocardial event and a region affected by the myocardial event.

According to an embodiment, a combination of features is proposed:
An ability to recognize MI events within the implant subject to scrutiny of the ST segment
In response to the recognition of MI events by the Implant, the subsequent further scrutiny of ST segment characteristics from a plurality of in-system sensing/pacing vectors
Leverage of the subsequent multi-vector ST segment scrutiny (i.e., “triangulation) to afford implant recognition of regions within the patient anatomy challenged by post-MI ischemia
Utilize the knowledge of the spatial locations of ischemic cardiac tissue to determine a sub-set potential sensing/pacing vectors removed from the influence and challenges of the ischemia
Supports a capacity to, in turn, evaluate the viability of this found sub-set of possible improved vector choices by running one or more follow-up tests (i.e., electrode, sensing, and pacing capture threshold tests) on each
Employing a sorting algorithm to rank the sub-set of vectors based on both the data gathered by their relevant follow-up tests but also subject to the influence of clinical guidance on how the system should best adapt in the event that a myocardial infarction occurs
The ability to ultimately modify the vector selection for sensing and pacing between follow-up in response to all of the above.

According to an embodiment, the found data is leveraged regarding the spatial localization of the heart tissue damage to, in turn, determine whether or not a preferred stimulation vector might better enable the evoked responses necessary for viable heart stimulation. In understanding which portions of the heart are compromised, the implant can recognize a subset of the total possible series of stimulation vectors that may have improved capacities for instating patient therapy support. A best mode of the invention would then take that subset of candidate vector options and, in turn, run electrode impedance, sensing, and pacing capture threshold tests on each. A sorting routine would then be run based on the found test results from each vector to pick the “best” vector for subsequent patient care.

The embodiment offers a means to more meaningfully and safely support patient needs following MI via direct intervention. It is expected that the adapted output described in the present disclosure will nominally amount to an interim or temporary therapy that is simply a “best the device can offer” approach until the patient is taken into a more capable clinical facility. Further, it is expected that a best embodiment would offer clinicians means for influencing how it behaves even to the extent of turning off any allowance for such adaptation if they estimate that doing so is in the best interest of the patient. Influence in the adaptation could involve boundary conditions where, for example, the clinician specifically bars the use of certain other in-system vectors as allowed options in the adaptive suite. Care providers would also be expected to have means for influencing how the ranking of the available alternative vectors occurs by, for example, demanding that the vector with the best evoked response during pacing capture tests are heavily weighted in the prioritization. A plurality of different criteria could be instituted at the direction of the care provider to coax any sort of post-MI adaptation to fully represent their preferences for the next best options in making an optimal vector selection.

The advantages and advantageous embodiments as described above for the system equally apply also to the method, such that it shall be referred to the above in this respect.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

The various features and advantages of the present invention may be more readily understood with reference to the following detailed description and the embodiments shown in the drawings. Herein,

FIG. 1 shows a schematic drawing of a system comprising an implantable medical device implanted in a patient;

FIG. 2 shows a schematic drawing of a patient's heart, with electrode leads of an implantable medical device arranged therein;

FIG. 3 shows a schematic drawing of an implantable medical device with electrode leads extending from a generator device;

FIG. 4 shows a drawing of an electrocardiogram signal;

FIG. 5 shows a drawing of a QRS complex of an electrocardiogram signal;

FIG. 6 shows an electrocardiogram signal over multiple cardiac cycles;

FIG. 7 shows a portion of an electrocardiogram signal representing a QRS complex with a subsequent ST segment elevation;

FIG. 8 shows a schematic drawing of an artificial intelligence model; and

FIG. 9 shows a schematic drawing of a system comprising an implantable medical device, the system being configured for outputting an alert on the occasion of identifying a myocardial infarction event;

FIG. 10 shows a quadripolar lead-based cardiac stimulation system;

FIGS. 11A-11C show a three-step sequence of a quadripolar lead-based cardiac stimulation system according to an embodiment

### DETAILED DESCRIPTION

Subsequently, embodiments of the invention shall be described in detail with reference to the drawings. In the drawings, like reference numerals designate like structural elements.

It is to be noted that the embodiments are not limiting for the invention, but merely represent illustrative examples.

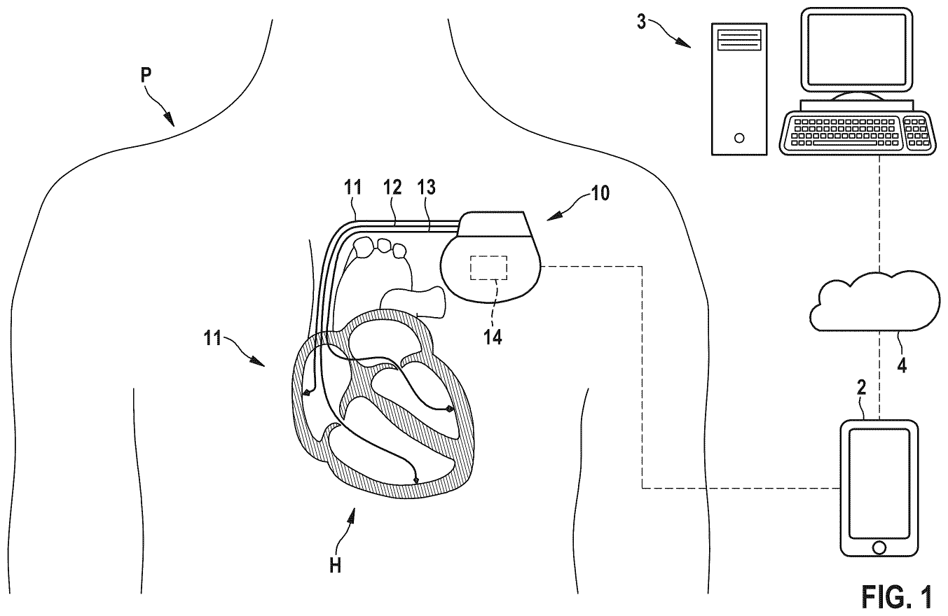

Referring to FIG. 1, in one embodiment a system comprises an implantable medical device 1 implanted into a patient for serving a therapeutic and/or diagnostic function. The implantable medical device 1, in the shown embodiment, comprises a generator device 10 and an arrangement of electrode leads 11, 12, 13 extending from the generator device 10. The generator device 10 may for example be implanted subcutaneously into a patient P, the electrode leads 11, 12, 13 reaching into the patient's heart H for monitoring cardiac activity of the patient's heart H.

The generator device 10 comprises a processing circuitry 14 encapsulated in a housing of the generator device 10 together with an electrochemical battery for supplying electrical energy for operation of the implantable medical device 1.

The system furthermore comprises an external device 2 external to the patient P and being in communication connection with the implantable medical device 1. The external device 2 may be in connection, via a public communication network 4, with a server device 3, for example a home monitoring service center, in the context of a home monitoring system.

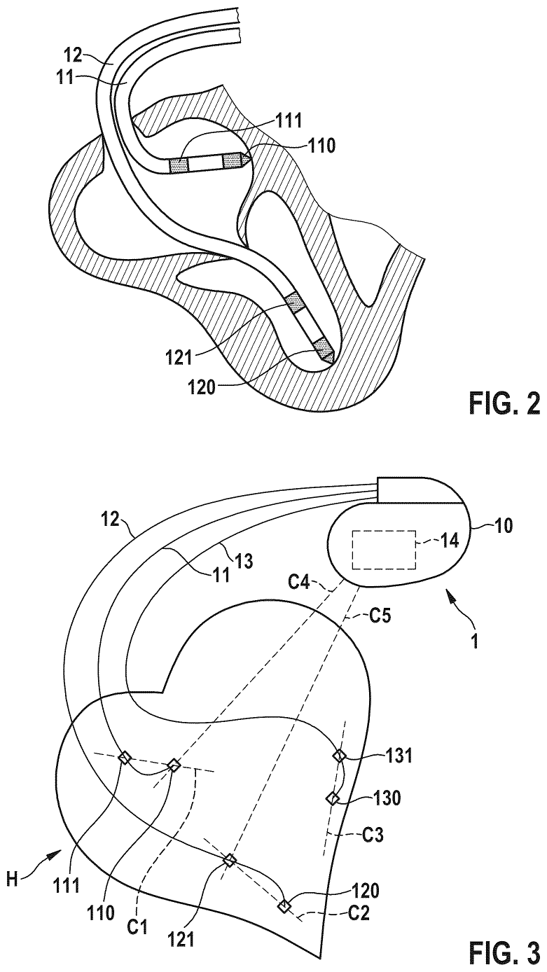

Referring now to FIGS. 2 and 3, in the shown embodiment each electrode lead 11, 12, 13 carries electrode poles 110, 111, 120, 121, 130, 131 for example formed by ring electrodes arranged on and circumferentially extending about a lead body of the particular electrode lead 11, 12, 13. In addition, an electrode pole may be formed by a housing of the generator device 10.

Using the arrangement of electrode poles 110, 111, 120, 121, 130, 131, electrocardiogram signals may be received and processed by the processing circuitry 14. Based on the processing, a communication with an external device 2 may be established, for example to transmit alert messages to the external device 2 and on to the server device 3 for example within the context of a home monitoring system for monitoring a physiological state of the patient P.

The different electrode poles 110, 111, 120, 121, 130, 131 of the electrode leads 11, 12, 13, optionally together with an electrode pole formed by the housing of the generator device 10, define signal reception vectors C1-C5 by means of which electrocardiogram signals may be received using pairs of associated electrode poles 110, 111, 120, 121, 130, 131. In particular, a first signal reception vector C1 may be formed between the electrode poles 110, 111 of the electrode lead 11 placed for example in the right atrium of the patient's heart H, a second signal reception vector C2 may be formed between the electrode poles 120, 121 of the electrode lead 12 placed for example in the right ventricle of the patient's heart H, a third signal reception vector C3 may be formed between the electrode poles 130, 131 of the electrode lead 13 placed for example in the left ventricle of the patient's heart H, a fourth signal reception vector C4 may be formed between the tip electrode 110 of the electrode lead 11 and the electrode pole formed by the housing of the generator device 10, and a fifth signal reception vector C5 may be formed by an electrode pole 121 of the electrode lead 12 and the electrode pole formed by the housing of the generator device 10. Further signal reception vectors may be spanned by other pairs of electrode poles.

The different electrode poles hence form different pairs of electrode poles spanning different signal reception vectors C1-C5. The signal reception vectors C1-C5 are oriented along different spatial directions and span across different spatial regions of the patient's heart H, such that by means of the different pairs of electrode poles electrocardiogram signals may be received in a spatially differentiated manner, one signal reception vector providing for an increased spatial sensitivity in a particular region compared to other signal reception vectors.

By means of the different signal reception vectors C1-C5, thus, different electrocardiogram signals may be received and may be processed in a multi-channel processing.

For example, any number between 2 and 16 signal reception vectors C1-C5 may be used to receive electrocardiogram signals, for example 12 signal reception vectors C1-C5 to receive a 12-channel ECG signal, similar to a 12-channel ECG device.

It shall be noted herein that the implantable medical device 1 may be a device having leads 11, 12, 13 carrying electrode poles 110, 111, 120, 121, 130, 131, or a device not having leads, in which case electrode poles may be arranged e.g. on a device housing, The implantable medical device 1 may in particular be a monitoring device, a pacemaker device, a defibrillator device or any other implantable medical device configured for implantation into a patient P.

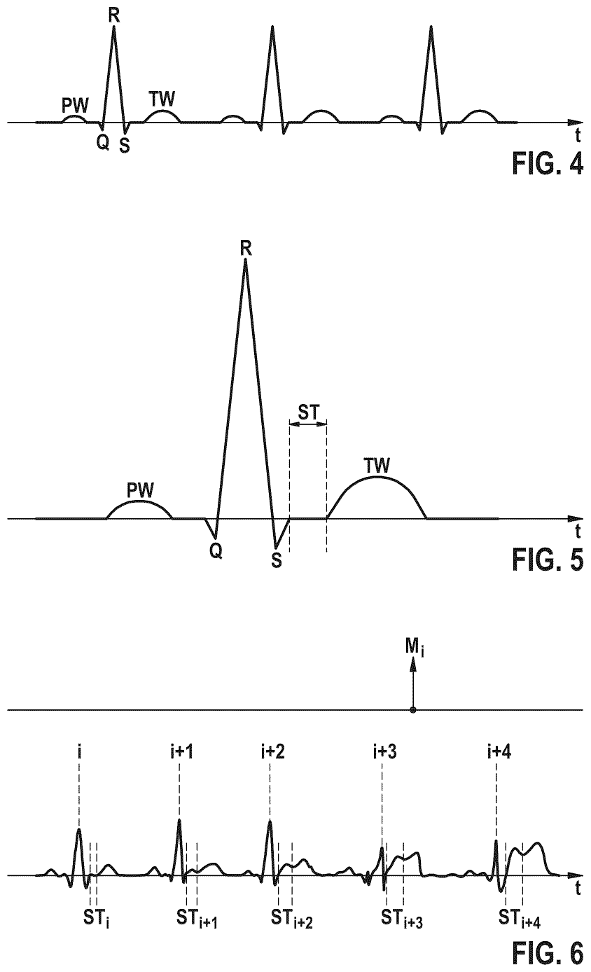

Referring now to FIG. 4, the system including the implantable medical device 1 and the external device 2 shall be configured for identifying a myocardial infarction event based on a processing of electrocardiogram signals, wherein the electrocardiogram signals may be received in different channels according to different signal reception vectors C1-C5 and may be processed in a multi-channel processing in order to derive information from the electrocardiogram signals enabling an identification of a myocardial infarction event.

Within an electrocardiogram signal, cardiac activity generally is represented by waveforms relating to atrial and ventricular activity. Within a cardiac cycle, a P wave PW represents the beginning of the atrial systole. The P wave PW is followed by the so-called QRS complex, the QRS complex generally forming a Q peak, an R peak and an S peak. The QRS complex is caused by ventricular depolarization, the QRS complex being followed by the so-called T wave TW representing ventricular repolarization.

As shown in FIG. 5, a signal portion in between the end of the QRS complex and the subsequent T wave TW is typically referred to as ST segment. The ST segment starts with the end of the S peak and ends with the beginning of the T wave TW, wherein in a healthy patient, i.e. a patient with a healthy cardiac state, the electrocardiogram signal in the ST segment is almost flat and close to the isoelectric baseline, as visible from FIG. 5.

Referring now to FIG. 6, the signal shape in the ST segment may change on the occurrence of a myocardial infraction, due to myocardial ischemia. In particular, it has been observed that upon the occurrence of a myocardial infarction an elevation or depression of the signal in the ST segment occurs, the ST segment hence substantially deviating from the isoelectric baseline.

In the example of FIG. 6, it may be observed that in cardiac cycles i . . . i+4 a signal alteration in the ST segment STᵢ . . . STᵢ₊₄ occurs, a substantial ST segment elevation being present in the cardiac cycles i+3, i+4. The ST segment elevation is indicative of a myocardial infarction MI, which correspondingly may be identified based on a signal analysis.

To identify a myocardial infarction event MI, it herein is proposed to record electrocardiogram signals using the implantable medical device 1 and the electrode arrangement formed by the electrode poles 110, 111, 120, 121, 130, 131. In particular, electrocardiogram signals as recorded using the implantable medical device 1, for example in a multi-channel processing of the signals of the different signal reception vectors C1-C5 in parallel, may be processed to analyze the ST segment of a cardiac cycle, wherein based on the processing a myocardial infarction event MI may be identified.

Referring now to FIG. 7, within the processing for example characteristic values may be derived from the electrocardiogram signal relating to the ST segment.

For example, the ST segment may be identified based on a timing with respect to the R peak of a prior QRS complex, wherein the ST segment may for example be assumed to start at a timing distance T1 with respect to the prior R peak (indicated by the maximum amplitude of the QRS complex) and may be assumed to have a duration T2. The timing distance T1 and the duration T2 may be predefined, wherein the timing distance may for example lie in a range between 0 ms and 150 ms and the duration T2 may lie in a range between 100 to 400 ms. The timing distance T1 and the duration T2 may be adapted during operation, for example dependent on the actual heart rate.

Based on the identification of the ST segment, characteristic values X1, X2, Xi may be determined, for example according to the elevation of the ST segment above baseline (characteristic value X1), according to the area under the signal curve relating to the ST segment (characteristic value X2), or according to signal values at certain points throughout the ST segment (characteristic values Xi). Alternatively or in addition, derivative values of a first-or higher-order derivative at certain points may be determined as characteristic values. Other characteristic values may be derived, based on defined mathematical metrics.

One, some or all of the characteristic values X1, X2, Xi may be compared to reference values in order to identify a myocardial infarction event MI, the reference values for example relating to a healthy cardiac state and being obtained according to electrocardiogram signals in a healthy state, for example upon initial implantation, wherein based on a deviation from the reference values a myocardial infarction event MI may be identified.

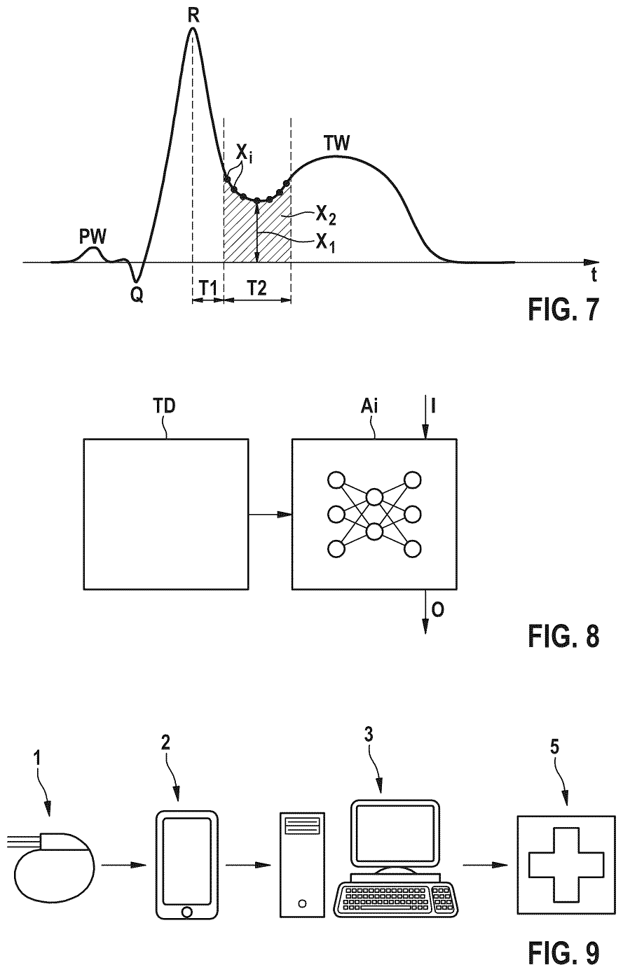

Referring now to FIG. 8, in one embodiment an artificial intelligence model AI may be used to identify a myocardial infraction event MI. In particular, characteristic values X1, X2, Xi as derived from an electrocardiogram signal may be input as input data I to the artificial intelligence model AI, and output data O may be output by the model AI, the output data O being indicative of the occurrence of a myocardial infarction event MI, for example of a score indicating the likelihood of the occurrence of a myocardial infarction event MI.

For defining the model, the artificial intelligence model AI may be fed with training data TD, the training data TD for example including electrocardiogram signals obtained from a large number of implantable medical devices implanted in a pool of patients for which myocardial infarction events have occurred and have been identified for example by the automatic processing of signals or by a confirmation of a physician correspondingly entered into the system. The training data TD may be collected over a large pool of patients, wherein the amount of training data TD may continuously grow with a growing population of implantable medical devices within the patients and a correlation of electrocardiogram signals with known and confirmed myocardial infarction events. For example, the implantable medical devices may relay information relating to electrocardiogram signals and myocardial infarction events (using a triggered, periodical or continuous reporting) to wearables, mobile devices, or bedside units that can pass data on to centralized repositories. Based on a data collection on those centralized repositories the artificial intelligence model AI may continuously be refined.

The artificial intelligence model AI may be defined in a training phase prior to operation of the system including the implantable medical device 1. The artificial intelligence model AI herein may be fixed, such that the model is not refined during actual operation, but is defined during the initial training phase and is not modified thereafter. In other embodiments, the artificial intelligence model AI may be self-learning in that data is constantly fed to the artificial intelligence model AI from the implantable medical device 1 and from a large variety of further implantable medical devices implanted in patients, such that based on a data collection of a variety of implanted medical devices the model AI is constantly modified and refined in order to improve its sensitivity and specificity.

The artificial intelligence model AI may be implemented on the implantable medical device 1. In other embodiments, the artificial intelligence model AI may be implemented on an external device 2 or on a server device 3, as schematically shown in FIG. 1, such that a substantial amount of processing takes place outside of the implantable medical device 1.

If the actual processing for identifying a myocardial infarction event takes place outside of the implantable medical device 1, the implantable medical device 1 may for example record electrocardiogram signals and may communicate information relating to the electrocardiogram signals to the external device 2 to provide signal data for the subsequent processing. The implantable medical device 1 herein may communicate the signal data as raw data to the external device 2, or may derive information, such as information relating to characteristic values X1, X2, Xi indicative of ST segment properties, to the external device 2.

Based on the processing, a monitoring of electrocardiogram signals for a myocardial infarction event may take place, wherein the ST segment for example in each cardiac cycle or periodically in one cardiac cycle per defined time period, for example once every 10 seconds, once every 30 seconds, once a minute, for instance, may be analyzed. Shorter time periods may allow for a close monitoring for the occurrence of a myocardial infarction event. Longer time periods may save computational and hence power resources.

In case a myocardial infarction event is identified, an alert action may be triggered. For example, an alert message may be sent to healthcare personnel in order to alert the healthcare personal of the myocardial infarction event at the patient.

Alternatively or in addition, an alert message may be sent for example to an emergency facility 5, as illustrated in FIG. 9, such that an emergency treatment may be provided to the patient. For example, a message to an emergency hotline, such as an emergency phone line (“911” in the US), may be triggered, such that emergency treatment may be provided to the patient. The sending of the alert message may be initiated by the server device 3, which receives information relating to an identified myocardial infarction event from an external device 2 that is in communication connection with the implantable medical device 1, or which receives electrocardiogram signal data via the external device 2 from the implantable medical device 1 and processes the data to identify a myocardial infarction event.

In addition, identified individuals, such as family members or friends of the patient, may be notified by alert messages.

An alert message may be triggered automatically in case of identification of a myocardial infarction event. Emergency attention hence may be provided to the patient without relying upon the presence and goodwill of bystanders to the patient, and without relying on a sufficient capacity of the patient to by herself trigger an alert in case of a myocardial infarction event.

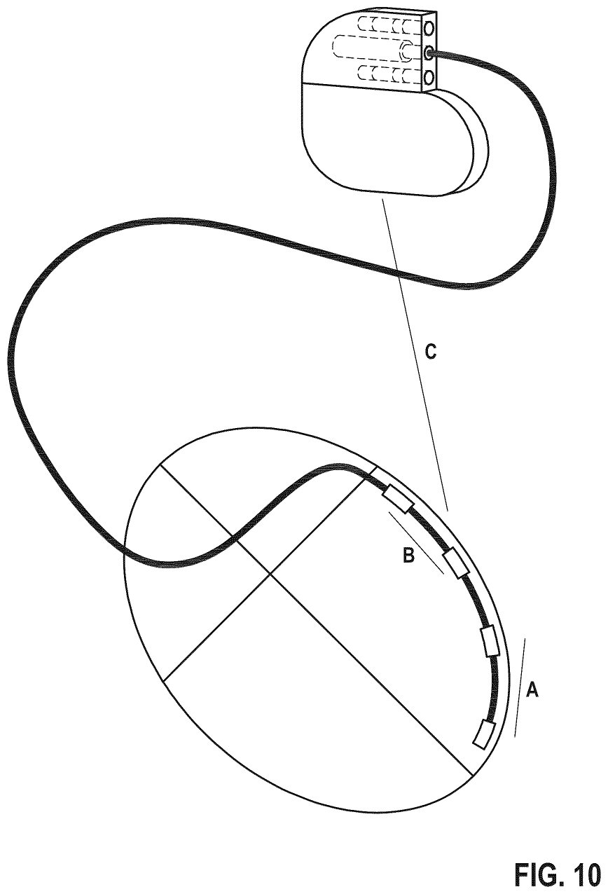

Referring to FIG. 10, an image indicative of how a multitude of vectors exist in a quadripolar lead-based system is shown. For simplicity, only the LV lead (blue) has been drawn, but it is expected that within such a system the LV lead would not be the lone lead involved in the administration of therapy. For further added simplicity, just a handful of vectors (i.e., pairs of electrodes) have been shown (A, B, and C) of the full suite of possible pair-wise electrode combinations.

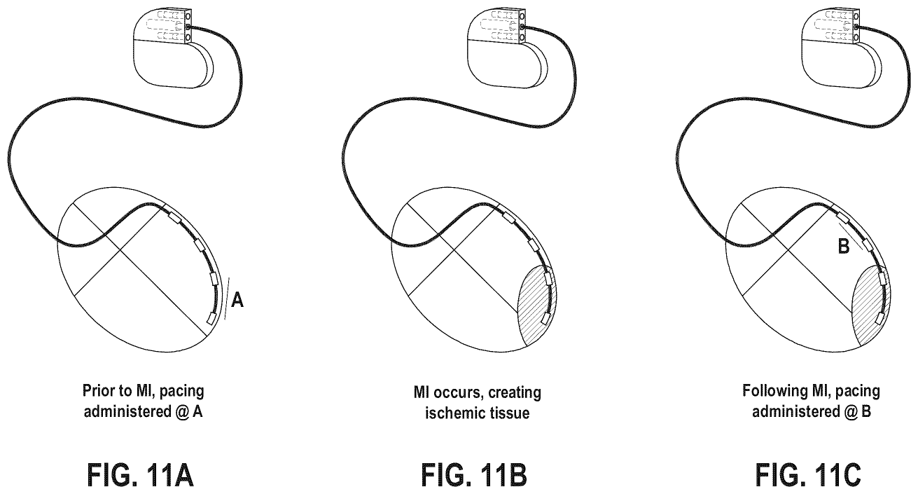

FIGS. 11A-11C show a three-step sequence to provide a visual that outlines the basic routine used to respond to an MI and adapt the pace output vector. As the MI occurs in step 11B, a routine is set off within the Implant to use “triangulation” to determine which portions of the patient heart are newly ischemic (dark gray shading in the center depiction). This routine runs evaluations on the sub-set of vectors outside of this compromised region (nominally executing the follow-up tests on each) to then rank order and select a new more appropriate vector in light of the recent cardiac tissue insult. In comparing step 11C to 11A in the shown example, the system adapts to administering therapy on vector B as opposed to the initial A vector that had been used prior to the MI-the latter now residing within an ischemic region of the heart.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

### LIST OF REFERENCE NUMERALS

1 Implantable medical device
10 Generator
11 Electrode lead
110,111 Electrode pole
12 Electrode lead
120, 121 Electrode pole
13 Electrode lead
130, 131 Electrode pole
14 Processing circuitry
2 External device
3 Server device
4 Public communication network
5 Emergency facility
AI Artificial Intelligence (AI) model
C1-C5 Signal reception vector
i . . . i+4 Cardiac cycle
I Input data
MI Myocardial infarction event
O Output data
PW P wave
P P wave
Q Q peak
R R peak
S S peak
ST, STᵢ . . . STᵢ₊₄ ST segment
TW T wave
T1, T2 Duration
TD Training data
TE Output
X1, X2, Xi Characteristic value

1. A system for performing a therapeutic and/or diagnostic function in a patient, comprising:
an implantable medical device configured for implantation in a patient, the implantable medical device comprising an electrode arrangement having a multiplicity of electrode poles for sensing an electrocardiogram signal; and
a processing arrangement for processing the electrocardiogram signal obtained by the electrode arrangement;
wherein the processing arrangement is configured to process the electrocardiogram signal to derive information from the electrocardiogram signal relating to an ST segment subsequent to an R peak of a QRS complex and prior to a T wave in a cardiac cycle and to identify a myocardial infarction event based on the information.

2. The system according to claim 1, wherein the implantable medical device comprises a processing circuitry which implements at least a portion of the processing arrangement.

3. The system according to claim 1, comprising an external device configured for operation external to the patient, wherein the implantable medical device is configured to communicate data relating to the electrocardiogram signal or relating to information derived from the electrocardiogram signal to the external device.

4. The system according to claim 3, comprising a server device configured to communicate with the external device for transferring information to and/or receiving information from the external device, wherein the server device is configured to process information derived from the electrocardiogram signal to identify a myocardial infarction event and/or to process information relating to an identified myocardial infarction event.

5. The system according to claim 1, wherein the multiplicity of electrode poles form at least two pairs of electrode poles, the at least two pairs of electrode poles defining different signal reception vectors.

6. The system according to claim 5, wherein the processing arrangement is configured to process signals obtained by the at least two pairs of electrode poles to derive, for each signal reception vector, information relating to an ST segment.

7. The system according to claim 6, wherein the processing arrangement is configured to identify a myocardial infarction event based on the information derived for the different signal reception vectors.

8. The system according to-or claim 1, wherein the processing arrangement is configured, for deriving the information relating to an ST segment, to identify a signal portion relating to an ST segment based on a timing with respect to a QRS complex in a cardiac cycle.

9. The system according to claim 1, wherein the implantable medical device is configured to store at least one of a signal portion relating to an ST segment and information derived from the electrocardiogram signal relating to an ST segment.

10. The system according to claim 1, wherein the processing arrangement is configured to determine, to derive the information from the electrocardiogram signal, a set of characteristic values relating to an ST segment from the electrocardiogram signal.

11. The system according to-one claim 1, wherein the processing arrangement is configured to identify a myocardial infarction event based on the electrocardiogram signal using an artificial intelligence model.

12. The system according to claim 11, wherein the processing arrangement is configured to train the artificial intelligence model using training data, wherein the training data includes information obtained from a multiplicity of implantable medical devices.

13. The system according to-one claim 1, wherein the processing arrangement is configured to trigger an alert action based on the identification of a myocardial infarction event.

14. The system according to claim 12, wherein the processing arrangement is configured to generate, as said alert action, an alarm message to an |emergency facility, said message including information for identifying the patient to the emergency facility.

15. A method for operating a system for performing a therapeutic and/or diagnostic function in a patient, the method comprising:
providing an implantable medical device configured for implantation in a patient, the implantable medical device comprising an electrode arrangement having a multiplicity of electrode poles for sensing an electrocardiogram signal; and
processing the electrocardiogram signal obtained by the electrode arrangement, including processing the electrocardiogram signal to derive information from the electrocardiogram signal relating to an ST segment subsequent to an R peak of a QRS complex and prior to a T wave in a cardiac cycle and identifying a myocardial infarction event based on the information.
